(19) United States
(12) Patent Application Publication
(10) Pub. No.: US 2020/0358925 A1
(43) Pub. Date: Nov. 12, 2020

(54) Health Monitoring System Including Privacy-Ensuring Obfuscated Camera Images

(71) Applicant: David R. HALL, Provo, UT (US)

(72) Inventors: David R. Hall, Provo, UT (US); Daniel Allen, Provo, UT (US); Conrad Rosenbrock, Provo, UT (US); Ben Swenson, Lehi, UT (US); Daniel Hendricks, Provo, UT (US); Andrew Nguyen, Provo, UT (US)

(21) Appl. No.: 16/962,661

(22) PCT Filed: Jan. 16, 2018
(86) PCT No.: PCT/US2018/013836

§ 371 Date: Jul. 16, 2020

(51) Int. Cl. H04N 1/44; H04N 7/18; H04N 5/225; A61B 5/00; A61B 5/01; A61B 1/227; A61B 1/267; A61B 1/303; A61B 1/307; A61B 1/313; A61B 1/24; A61B 1/247; A61B 3/12; A61B 5/0205; A61B 5/103; A61B 5/11; A61B 5/107; A61B 3/117; H04N 1/32; A61B 3/14; G06F 21/62; H04L 9/06

(52) U.S. Cl. CPC H04N 1/448; A61B 5/02427; H04N 5/2256; H04N 7/188; A61B 5/0013; A61B 5/0077; A61B 5/0064; A61B 5/015; A61B 1/227; A61B 1/267; A61B 1/303; A61B 1/307; A61B 1/313; A61B 1/24; A61B 1/247; A61B 3/12; A61B 5/02055; A61B 5/1032; A61B 5/1128; A61B 5/4561; A61B 5/1077; A61B 5/1079; A61B 5/444; A61B 5/448; A61B 5/4878; A61B 5/418; A61B 3/1176; H04N 1/32283; A61B 3/14; G06F 21/6245; H04L 9/0643; H04N 2201/0084; H04N 7/181

(57) ABSTRACT

The unobtrusive health-monitoring apparatus includes a camera, a controller, a data transmission port, and, optionally, a graphics processing unit (“GPU”) which executes nonlinear transformation algorithms. The camera may be inconspicuously disposed within a fixture in the room. The camera collects graphic data which may include still images or video of the user, or both. The GPU may execute the nonlinear transformation algorithms which may transform the graphic data into formats which cannot be recognized by humans. However, these formats preserve features that can be parsed by machine learning methods and used for health tracking purposes. The formats cannot be parsed by humans or be converted back to the original image or video by mathematical inversion or available computational methods. User privacy is thus preserved. The graphic data may be transmitted to a remote processor. The remote processor may perform algorithms which create a health status analyses.

### CROSS-REFERENCE TO RELATED APPLICATIONS

This application claims priority to PCT International Patent Application Number PCT/US2018/13836 titled “Health Monitoring System Including Privacy-Ensuring Obfuscated Camera Images” filed on 16 Jan. 2018, which is incorporated herein by reference in its entirety.

### TECHNICAL FIELD

This disclosure relates to apparatus for assessing a user's health status by analyzing collected data.

### BACKGROUND

Behavior and body motion are key indicators of both mental and physical health. Images of an individual may reveal changes in skin color and areas of unusual pigmentation, changes in body composition, changes in the individual's gait, and behavioral patterns. Examples of conditions which may be identified by assessing color on a patient include cyanosis, anemia, jaundice, rashes, skin lesions, and skin cancers. Examples of conditions which affect movement and behavior include depression, stroke, tremors, joint damage, fatigue, anxiety, and many more. Theoretically, analysis of video and still photos collected in an individual's home or care center could offer the possibility of early diagnosis for many conditions. However, health tracking and monitoring of patients at home by still photo and video poses both security and privacy concerns, particularly when transmitting the data to a remote healthcare provider.

Analysis of still photos and video could be analyzed using telemedicine techniques. A goal of telemedicine is to provide continuous unobtrusive monitoring of users and early alert, detection, and monitoring of progression of health conditions. Machine learning algorithms can recognize users from still images and video and classify behavior, movement, morphology, coloring, and other observations. However, many users may feel uncomfortable with being observed continuously by video in a home, work, or care center environment. In addition, collecting such data may represent a safety concern. Furthermore, video image of users, if intercepted, could pose a threat to security. In an example, video of someone typing a password into their home computer could result in theft. A health-monitoring system is needed which collects visual data of a user in the user's natural environment while safeguarding the user's privacy, safety, and security.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; and US 2018/0165417, entitled “Bathroom Telemedicine Station.” The disclosures of all these patents and applications are incorporated by reference in their entireties.

### SUMMARY

We disclose a health-monitoring apparatus which discretely and unobtrusively collects graphic data. This graphic data may be used to assess the user's health status. The apparatus may include a camera which may be positioned such that it may collect graphic data of a user. The apparatus may include a controller which has a memory and a communications port. The camera may be in electronic communication, either wired or wireless, with the controller. A memory within the controller may store instructions for transmitting graphic data to a remote database, for example, a cloud database. The remote database may store non-transitory computer-readable media which includes instructions for applying an algorithm, in some embodiments, a machine-learning algorithm, to the graphic data. The algorithm may create an analysis of the graphic data and an assessment of a user's health status. In some embodiments, the algorithm also prepares a report describing the status of the user's health and transmits it to the user or the user's healthcare provider.

The apparatus may include a light source which may direct light toward the camera's angle of view. More specifically, the light source may direct the light toward the user as the camera collects graphic data including images and video of the user. Depending on the type of health assessment to be made, the light may include a defined range of wavelengths and patterns of light. Examples of ranges of wavelengths and patterns of light include, but are not limited to, visible light, high color temperature light, infrared light, structured light, and modulated light.

A variety of types of cameras may be included in the apparatus. The apparatus may include more than one camera and more than one type of camera. Examples of types of cameras which may be included in the apparatus include, but are not limited to, a 3D time of flight camera, a stereoscopic camera, an infrared thermal imaging camera, a video camera, a structured light 3D scanner, and a still image camera. These different cameras may collect different graphic data which may have different diagnostic uses. For example, the apparatus may use different cameras and different types of light to assess the speed of user movement, user movement patterns, user posture, swelling beneath the user's eyes, swollen lymphatic glands, visual photoplethysmography, heart rate, moles, skin growths, body shape, skin coloration, sclera coloration, hair loss, breathing rate, time in front of the camera, and body shape.

In addition to a fixture, the camera may be disposed within a variety of diagnostic tools. These may include an otoscope, an ophthalmoscope, an endoscope, a laparoscope, a laryngoscope, a colposcope, a hysteroscope, a bronchoscope, a pharyngoscope, a laparoscope, or a dental tool.

In some embodiments, the apparatus includes an auxiliary sensor which detects the presence of a user and actuates the camera, the light source, or both to collect graphic data relating to the user. In some embodiments, the auxiliary sensor is a motion detector. In some embodiments, the auxiliary sensor is a pressure sensor. In some embodiments, the pressure sensor is a floor scale which the user may stand on as the user approaches the camera.

In some embodiments the controller includes a graphics processing unit which includes instructions for performing multiple nonlinear transformations on the graphic data the camera collects. These transformations convert the graphic data to obfuscated graphic data representing obfuscated images which a human view cannot recognize as the image or video originally collected by the camera. However, the obfuscated graphic data retains at least one feature that a machine-learning algorithm can recognize. Non-transitory computer-readable medium stored on the remote database may include instructions for applying a machine-learning algorithm to the transformed graphic data without reconstructing the original image or video. The machine-learning algorithm may create an analysis of the obfuscated graphic data and an assessment of the user's health status.

Examples of techniques which may be used to transform the graphic data collected by the camera into obfuscated graphic data include deep convolution, compressed sensing obfuscation in which a truncated sparse basis expansion is used, and block-chain based obfuscation. In the block-chain based obfuscation technique, difference hashing between frames may be secured via blockchain.

By transmitting the data to the remote server as obfuscated graphic data, the user's privacy is preserved. In addition, the safety and security of the transmitted graphic data is enhanced relative to transmitting non-transformed graphic data.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood regarding the following description and accompanying drawings where:

FIG. 1 illustrates an embodiment of the disclosed apparatus including a camera behind a partially silvered mirror and a motion sensor.

FIG. 2 illustrates another embodiment of the disclosed apparatus in which a camera is disposed within a ceiling light fixture.

FIG. 3 illustrates an embodiment of the disclosed apparatus including a camera behind a partially silvered bathroom mirror and a floor scale.

FIG. 4A illustrates a still image of a cat which may be collected by a camera which is part of an embodiment of the disclosed apparatus.

FIG. 4B illustrates the image of FIG. 4A which has been transformed to obfuscate the image of the cat according to an embodiment of the disclosure.

FIG. 4C illustrates the results of attempts to reconstruct the image of FIG. 4A from the obfuscated graphic data of FIG. 4B.

FIG. 5A illustrates an image of a hashtag (#) which may be collected by a camera which is part of an embodiment of the disclosed apparatus.

FIG. 5B illustrates the image of FIG. 5A which has been transformed to obfuscate the image of the hashtag (#) according to an embodiment of the disclosed apparatus.

FIG. 5C illustrates the result of an attempt to reconstruct the image of FIG. 5A from the obfuscated graphic data of FIG. 5B.

FIG. 6A illustrates a still image of a woman which may be collected by a camera which is part of an embodiment of the disclosed apparatus.

FIG. 6B illustrates the image of FIG. 6A which has been transformed to obfuscate the image according to an embodiment of the disclosed apparatus.

FIG. 7 illustrates a flow chart showing steps which may be taken to use an embodiment of the disclosed apparatus.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “safe” means that data which could reasonably be exploited by actors with malicious intent is prevented from being accessed by such actors or their agents.

As used herein, “secure” means only authorized parties receive data.

As used herein, “private” means the data is kept exclusively within the health monitoring system to which the user has subscribed.

As used herein, “user” means the individual from whom the disclosed system is collecting graphical data.

As used herein, “electronic” means either wired or wireless. For example, the phase “in electronic communication” means either a wired communication between two devices or could mean a wireless communication between devices, such as WiFi.

### Exemplary Embodiments

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a system for discrete and unobtrusive health monitoring using video, still photos, or both. The system may be used in the home, care facility, or clinical setting. The system may include one or more cameras and a controller. The controller may include a memory and be in electronic communication with the camera. The electronic communication may be either wired or wireless. The one or more cameras may collect graphic data and transmit the graphic data to the controller.

The controller may include a memory which may house non-transitory computer-readable medium. The non-transitory computer-readable medium may include instructions for applying an algorithm, which in some embodiments is a machine-learning algorithm, to create an analysis of the graphic data. The algorithm may further convert the analysis of the graphical data to an assessment of the user's health status. Alternatively, separate algorithms may perform the tasks of directly analyzing the graphic data and creating an assessment of the user's health. An algorithm may also create a report of the assessment of the user's health and transmit the report to the user or the user's healthcare provider.

The controller may also include a communication port which is in electronic communication with the controller, either wired or wirelessly. The controller may include non-transitory computer-readable medium which includes instructions to transmit the graphic data through the communications port. The communication port may transmit the graphic data to a remote server, for example, a cloud computing resource. Graphic data may be stored on the remote server. The remote server may store and implement non-transitory computer-readable medium which includes instructions for applying one or more machine-learning algorithms to create an analysis of the graphic data and to create an assessment of a user's health status. In some embodiments, the remote server is secure, safe, private, or a combination thereof.

Some embodiments may include a light source and a light source modulator. The light source modulator may direct light toward an angle of view of the camera. The light provided by the light source may enable the camera to collect better quality still or video images which are more readily analyzable. The modulator may select the wavelength of light or other characteristics of the light provided by the light source. For example, the light source may provide one or more of the following types of light: visible light, high color temperature light, infrared light, structured light, and modulated light. In an example, visible light may be useful to illuminate the subject to record movement and behavior. Controlled wavelength illumination may allow a degree of depth profiling. Infrared illumination may reveal vascular structure deeper in the skin. High color temperature (blue-tint) light may be useful to detect features on the skin surface. Infrared illumination may be used to record the user's motion in an otherwise unlit environment. Stereoscopy, time of flight, or structured lighting may be used to collect 3D information about the user.

The camera may be a single camera. Alternatively, the system may include a plurality of cameras. Each camera may include, but is not limited to, one or more of the following types of cameras: a 3D time of flight camera, a stereoscopic camera, an infrared thermal imaging camera, a video camera, a structured light 3D scanner, and a still image camera.

In an example, a camera is disposed within an otoscope, an ophthalmoscope, an endoscope, a laparoscope, a laryngoscope, a colposcope, a hysteroscope, a bronchoscope, a pharyngoscope, a laparoscope, or a dental tool. In some examples, the camera in these devices is a perspective camera.

In some embodiments, the camera may be disposed within a fixture within the room. By disposing the camera within a fixture, the camera is unobtrusive to the user and therefore, unlikely to cause the user to modify his or her behavior due to self-consciousness when the camera is collecting graphic data. In an example, the fixture may be placed in a bathroom, a kitchen, a hallway, an entryway, an office, or a living room.

In an example, the fixture includes a partially silvered mirror. The mirror may be disposed between the user and the lens of the camera (in front of the lens of the camera). The user may view himself or herself in the mirror in a traditional manner while the camera collects graphic data from the user. The mirror may appear no differently to a user than a traditional mirror. This characteristic may prevent the user from behaving differently because the user is focusing on the fact that the camera is collecting graphical data. In another example, a small window is disposed in the fixture and over the camera lens so that the camera may collect graphic data on the other side of the fixture.

In addition to the one or more cameras, the system may include one or more auxiliary sensors, each which may be in electronic communication with the controller. The non-transitory computer-readable medium on the controller may include instructions to actuate the health-monitoring apparatus when the controller receives a signal from an auxiliary sensor. In an example, an auxiliary sensor may be a pressure sensor. In a more specific example, the pressure sensor may be placed on or within flooring in the environment surrounding the one or more cameras. In some embodiments, the pressure sensor may be a floor scale or pressure sensitive floor mat. When a user enters the environment and crosses over the pressure sensor, the pressure sensor may detect the user's mass and send a signal to the controller. In another example, the auxiliary sensor may be a motion detector which sends a signal to the controller when it detects the presence of a user in the environment. In the embodiments which include an auxiliary sensor, the user does not need to consciously actuate the system. The system's presence may, therefore, be less noticeable. Consequently, the user may be less likely to modify his or her behavior due to self-consciousness about being recorded by the camera.

In some embodiments, the system may include an interactive display for the user. In the embodiment which includes a mirror, the display may appear on the mirror. The display may provide health data to the user, as measured in real time (such as the measured weight from the floor scale), as well as health data which is the result of the analysis performed by the system. The display may also include indicators of when images are being captured and when the process has completed. The display may also be connected to other information sources, such as a smart home system, and may be used to display non-health related data, such as weather, schedule or news.

The one or more cameras may record a plurality of clinically relevant observations. These may include behaviors and movements, for example, the speed of the user's movement, the patterns of the user's movements, the user's posture, the time of day that the user appears in front of the camera, uncharacteristic use of the non-dominant hand, and the amount time the user spends in front of the camera. Observations relating to behavior and movement may assist in the diagnosis of psychological, neurological, skeletal, and motor aberrations.

The one or more cameras may also record physiological changes in a user including, but not limited to, edema including swelling beneath the user's eyes, swollen lymphatic glands, visual photoplethysmography, heart rate, moles, skin growths, skin coloration, sclera coloration, mucous membrane coloration, coloration of the ear canal, hair loss, breathing rate, and body shape.

In an example, the camera may be disposed within an otoscope and collect graphic data which may be used to identify inflammation within the external auditory canal.

In another example, the camera may collect graphic data which may be used to assess the transparency of the lens of the user's eye. If the lens is relatively opaque, the assessment of the user's health status may indicate a possible cataract. Scratches and abrasions on the user's eye may also be detected. In such embodiments, the camera may be disposed within an ophthalmoscope or behind a mirror. In the example in which the camera is disposed behind a mirror, the mirror may be a bathroom mirror which the user spends time standing directly in front of and facing while performing toiletry tasks, for example, hair and teeth brushing. Consequently, the camera may be able to collect images of the user's eyes as the user peers directly into the mirror.

In an example in which the camera is disposed within a fixture, for example, behind a mirror, the camera may detect coloration in the user's skin. A yellow coloration may indicate jaundice while a blue coloration may indicate cyanosis. A pale coloration may indicate pallor which is a symptom of emotional stress, anemia, and other pathologies. The camera may measure the color and shape of a mole or skin growth leading to a dermatological diagnosis. The camera may record movement in the user's chest which may be used to calculate the user's breathing rate.

In some embodiments, the controller further includes a graphic processing unit (hereinafter, “GPU”). The GPU may include non-transitory computer-readable medium which includes instructions for performing algorithms which transform and obfuscate the graphic data the camera collects. The algorithms may perform multiple convolutions or nonlinear transformations on the graphic data which convert the graphic data to a form which is unrecognizable by a human viewer, but which also retains a feature recognizable by the machine-learning algorithm. The graphic data may be transformed into several kinds of transformed data by several different transformations, each transformation preserving a feature recognizable or useful for a machine-learning or computer classification algorithm. The graphic data is now obfuscated graphic data. In addition to being unrecognizable by humans, the obfuscated graphic data may not be converted back to the original graphic data by mathematical inversion or available computational methods. Examples of techniques which may be used to transform the graphic data include deep convolution, compressed sensing obfuscation using sparse basis expansion and discarding basis functions; and block-chain based obfuscation. In the block-chain obfuscation technique, difference hashing between frames may be secured via blockchain.

Obfuscation may be used to preserve safety, security, and user privacy. After the graphic data has been transformed, it may be transmitted to a remote processor which may apply algorithms to assess the status of the user's health. Because the graphic data has been transformed as described herein, the system maintains the user's privacy and the safety and security of the data is enhanced relative to graphic data which has not been transformed.

An example of an obfuscating, non-linear transformation is a multi-layer convolution network with non-linear pooling layers. A series of 2D kernels may be applied to each 2D frame of a video or still image collection using convolution, possible varying the padding and step size. This may produce a set of convolved images for each image (one for each kernel). At this point, it is still possible for an inverse convolution to reconstruct the original image. However, a pooling layer is then applied whereby the size of each convolved image is reduced by taking the average or maximum absolute value of all pixels within a neighborhood; for example, a group of 4 pixels may be replaced by a single pixel that has the maximum value of any of the original 4 pixels. This step is non-linear and cannot be exactly inverted. Typical implementations may have an alternating succession of 2D convolution and pooling layers to produce a final set of convolved images that are flattened to a single vector. This vector may encode information that may be useful to a machine-learning algorithm, but will appear to be random noise to a human viewer.

Another approach to obfuscating the video stream involves expanding each video frame using a sparse representation (for example, wavelet or discrete cosine) using an algorithm which may include compressed sensing techniques. These sparse regression algorithms penalize solutions to have a minimal l_1 norm, which preferentially selects sparse solutions. Then, machine-learning algorithms may be given access to data in the transformed space to train on. Only those basis functions that are relevant to the machine learning model are selected, allowing a large number of basis functions to be discarded. This loss of basis function renders the images unrecognizable.

Another way to obfuscate graphic data collected by the camera within the disclosed system is using a blockchain-based system. In this example, the user and server exchange public keys. By giving the server the user's public key, the user is granting permission for the server to access the obfuscated video stream. A difference hash is computed for each frame in the video relative to its neighboring frames. This hash is then combined with the user's private key and the server public key to generate a transaction in a blockchain ledger. Each frame captured and sent to the server is traceable to a transaction that was approved by the user for use by the server. Only the user or the server possess the keys to decrypt the difference hash in the video frames.

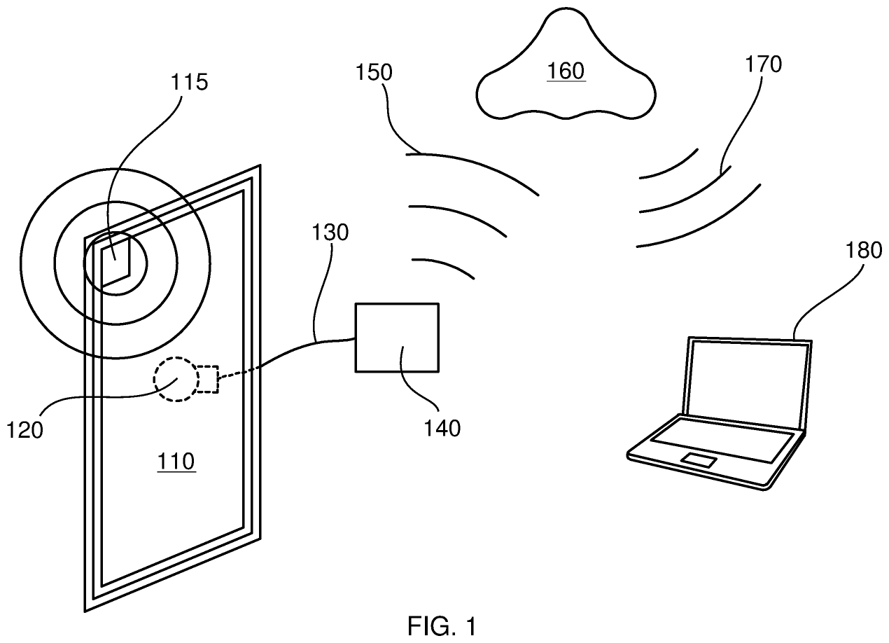

Referring now to the drawings, FIG. 1 illustrates an embodiment of the disclosed system which includes mirror 110 which is partially silvered. Mirror 110 has motion detector 115 incorporated into it. Motion detector 115 senses the presence of a user approaching mirror 110 and transmits a signal to camera 120 which is disposed behind mirror 110. Camera 120 collects graphic data in the form of still photos, video, or both through mirror 110. This graphic data may represent movement patterns, morphology, lesions, and coloring associated with a user who is present in front of mirror 110. Camera 120 can collect graphic data of people and objects on the other side of mirror 110 because mirror 110 is only partially silvered. Camera 110 transmits graphic data through wire 130 to controller 140. In other embodiments, camera 110 may transmit the graphic data through wireless technology. Controller 140 includes a memory which stores instructions for transforming graphic data so that it is obfuscated as disclosed herein. Controller 140 transmits the obfuscated graphic data through wireless signal 150 to remote database 160. A memory on remote database 160 stores instructions for using a machine-learning algorithm to analyze the obfuscated graphic data to assess the user's health status. The instructions also include steps for creating a report describing the user's health status. Wireless signal 170 then transmits the report to a user or the user's healthcare professional.

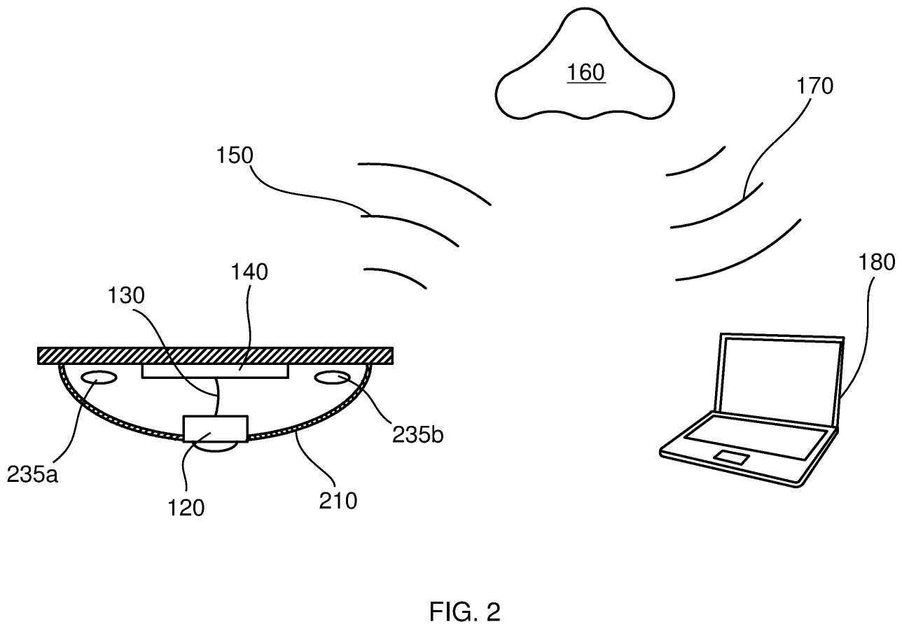

FIG. 2 illustrates yet another embodiment of the disclosed system which shows a cross-section of ceiling light fixture 210. Camera 220 is disposed within ceiling light fixture 210 and includes a lens which is directed generally downward towards a user who may entire the room below. Camera 220 collects graphic data which includes still and video images of the user in the room below ceiling light fixture 210. Camera 220 transmits the graphic data through wire 130 to controller 140. Controller 140 includes a memory which stores instructions for transforming graphic data so that it is obfuscated as disclosed herein. Controller 140 transmits the obfuscated graphic data through wireless signal 150 to remote database 160. In some embodiments, the graphic data may not be transformed before computer 140 transmits the graphic data to remote database 160. A memory within remote database 160 stores instructions for using a machine-learning algorithm to analyze the obfuscated graphic data to assess the user's health status. The instructions also include steps for creating a report describing the user's health status. Wireless signal 170 then transmits the report to a user or the user's healthcare professional.

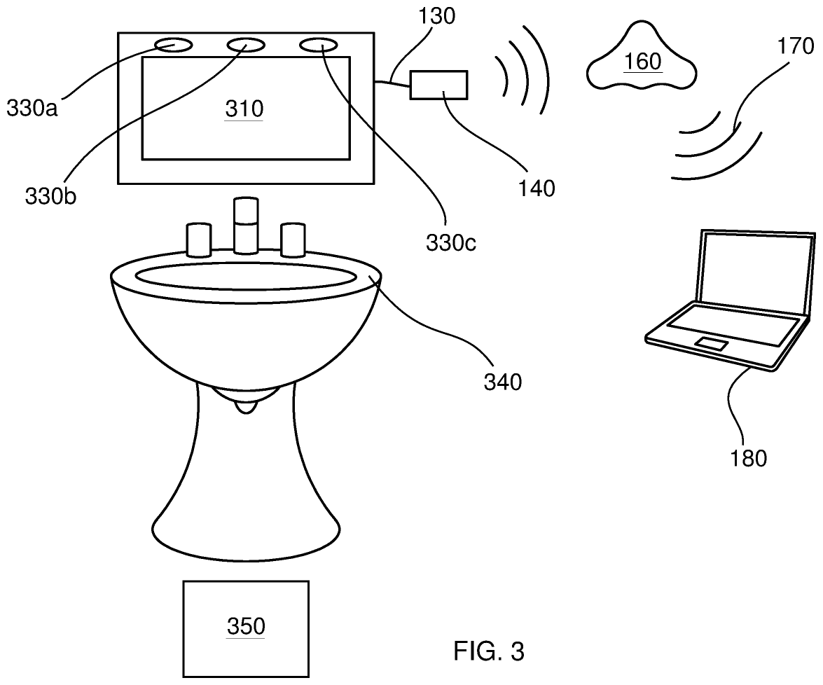

FIG. 3 illustrates yet another embodiment of the disclosed system which includes bathroom mirror 310. Bathroom mirror 310 is partially silvered so that a camera disposed behind bathroom mirror 310 may collect graphic data of a user standing in front of bathroom mirror 310. Light sources 330a, 330b, and 330c each emit light of a defined range of wavelengths and patterns toward a user standing in front of bathroom mirror 310. In an example, each of light sources 330a-c may emit a different range of wavelengths of light. In an example, the range of wavelengths of light may be in the infrared range or the visible range. A user may approach bathroom mirror 310 and step onto floor scale 350 which then sends a signal to actuate the camera behind bathroom mirror 310 and light sources 330a-c. The user may conduct typical activities using lavatory 340, for example, washing and teeth brushing, while the camera collects graphic data of the user. This graphic data may include still photos, video, or both. The camera transmits the graphic data through wire 130 to controller 140. Controller 140 includes a memory which stores instructions for transforming graphic data so that it is obfuscated as disclosed herein. Controller 140 transmits the obfuscated graphic data through wireless signal 150 to remote database 160. In some embodiments, the graphic data may not be transformed before computer 140 transmits the graphic data to remote database 160. A memory on remote database 160 stores instructions for using a machine-learning algorithm to analyze the obfuscated graphic data to assess the user's health status. The instructions also include steps for creating a report describing the user's health status. Wireless signal 170 then transmits the report to a user or the user's healthcare professional.

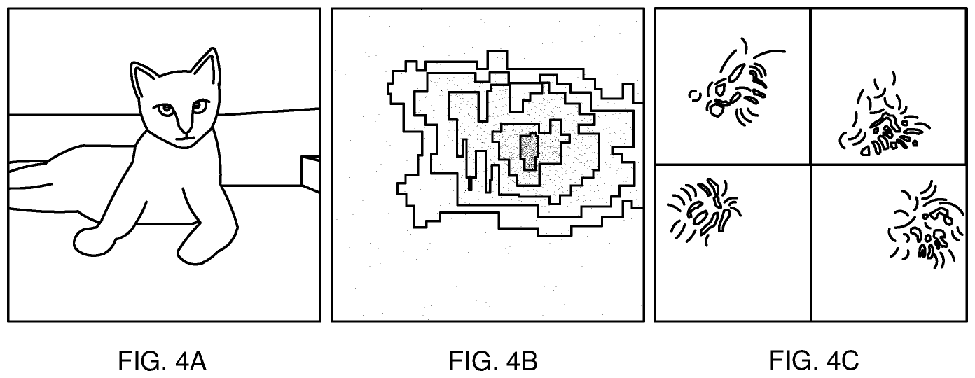

FIG. 4A illustrates an image of a cat which a human user may recognize, and which may be created using a camera according to the disclosed system. FIG. 4B shows the image of FIG. 4A as it may appear after the graphic data has been transformed using techniques disclosed herein. FIG. 4C illustrates what the best reconstruction of the image of FIG. 4A might look like. In FIG. 4C, the cat as shown in FIG. 4A is not discernable to a human viewer. Information about the machine-learning model would be necessary to reconstruct the image in FIG. 4A. A separate transformation is required for each quantity that a machine-learning model would predict, and each machine-learning model would find different parts of an image relevant for its prediction. Consequently, the image shown in FIG. 4A is nearly impossible to reconstruct from that shown in FIG. 4B. For the machine-learning models to be effective, there is a minimal amount of relevant data that must be encoded to make a decision. Although the non-linear transformations are not exactly invertible, they are approximately invertible as shown in FIG. 4C.

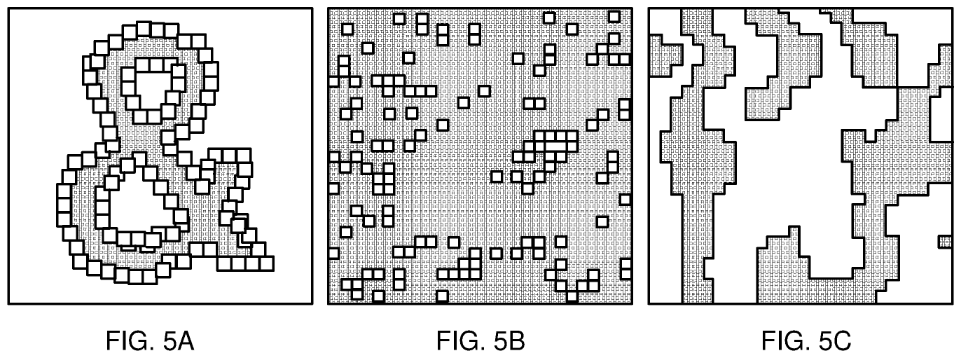

FIGS. 5A-5C illustrate an example of obfuscation via compressed sensing in a discrete cosine basis. FIG. 5A shows an ampersand (&). FIG. 5B shows a fraction of the image in FIG. 5A that may be selected as necessary for the basis reconstruction in FIG. 5C. While FIG. 5C has sufficient information content for a deep network to identify the ampersand (&), a human viewer cannot recognize the original ampersand (&) in the image.

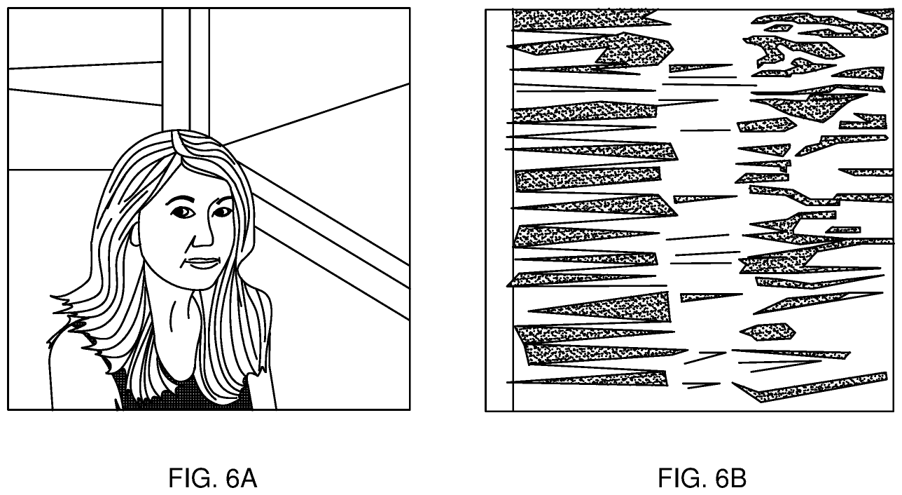

FIGS. 6A and 6B illustrate an example of obfuscation via encrypted difference hashing. FIG. 6A shows the original image of a woman. FIG. 6B illustrates an example of how the image in FIG. 6A, after being obfuscated, may look to a user who does not have the relevant keys. A human viewer who does not have access to either the server or user key that generated a transaction in the blockchain for that frame, will not be able to recover the original image.

Should the image in FIG. 6A be collected by a camera included in the disclosed system, there would be is an immutable public record of the frame being shared with a particular target (the remote server). However, once the graphic data associated with the image in FIG. 6A has been transformed (obfuscated) as described herein, the contents of the record are only decryptable by either the user, or the server. Once the hashes have been decrypted, the changes in the video stream can be reconstructed. Note that the original image shown in FIG. 6A is never uploaded to a server according to the instant disclosure. The machine learning algorithms look only at the differences from frame to frame and compare them over time to create predictive models.

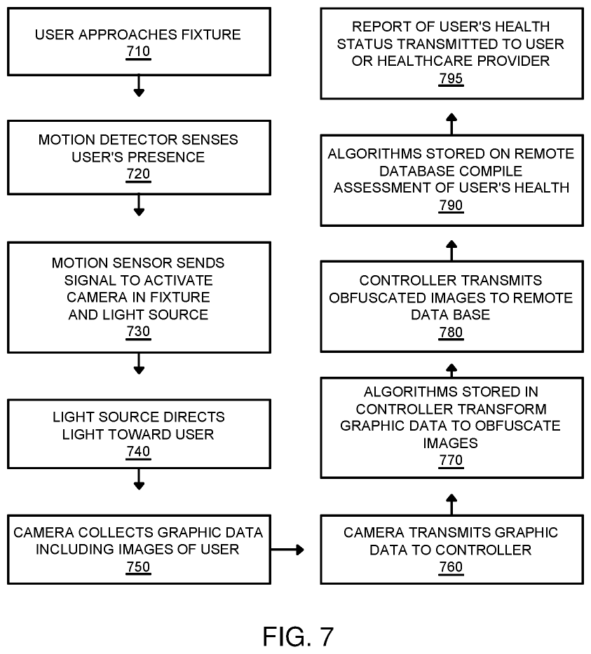

FIG. 7 is a flow chart illustrating steps which may be undertaken to use an embodiment of the disclosed system to assess a user's health status. In step 710 the user approaches a fixture which includes a camera according to the present disclosure. A motion detector within the system detects the user's presence (step 720) and actuates the camera and light source (step 730). In step 740, the light source directs light of a defined range of wavelengths toward the user. In an example, the light may be infrared or may be in the visible range. The camera then collects graphic data which includes images of the user (step 750). The graphic data may include video, still photos, or both. The camera may then transmit the graphic data to a local controller (step 760). In this embodiment, the controller includes a memory which stores instructions for performing algorithms which transform the graphic data to obfuscate the graphic data (step 770). In step 780, the controller then transmits the obfuscated graphic data to a remote database, for example, a cloud database. The remote database stores instructions which perform algorithms which use the obfuscated graphic data to assess the user's health status (790). The instructions then create a report of the user's health status and transmit it to the user or to the user's healthcare professional (step 795).

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

What is claimed is:

1. A health-monitoring system comprising:
at least one camera positioned to capture graphic data from a user;
a controller; the controller comprising a memory, wherein the controller is in electronic communication with the at least one camera, wherein the controller receives graphic data from the at least one camera;
a communication port, wherein the communication port is in electronic communication with the controller, and wherein the controller comprises instructions to transmit the graphic data through the communication port; and
non-transitory computer-readable media comprising instructions for applying an algorithm to transform the graphic data into obfuscated graphic data, to create an analysis of the obfuscated graphic data, and to create an assessment of a user's health status.

2. The health-monitoring system of claim 1, further comprising a light source, wherein the light source directs light toward an angle of view of the at least one camera.

3. The health-monitoring system of claim 2, wherein the light source provides light which consists of one or more of the following types of light: visible light, high color temperature light, infrared light, structured light, and modulated light.

4. The health-monitoring system of claim 1, wherein the at least one camera comprises of one or more of a 3D time of flight camera, a stereoscopic camera, an infrared thermal imaging camera, a video camera, a structured light 3D scanner, and a still image camera.

5. The health-monitoring system of claim 1, wherein the at least one cameras is disposed within a diagnostic tool, wherein the diagnostic tool consists of one or the following: an otoscope, an ophthalmoscope, an endoscope, a laparoscope, a laryngoscope, a colposcope, a hysteroscope, a bronchoscope, a pharyngoscope, a laparoscope, and a dental tool.

6. The health-monitoring system of claim 1, wherein the at least one camera is disposed within a fixture.

7. The health-monitoring system of claim 6, wherein the fixture comprises a partially silvered mirror, wherein the mirror is disposed in front of a lens of the at least one camera.

8. The health-monitoring system of claim 1, further comprising one or more auxiliary sensors, wherein the one or more auxiliary sensor is in electronic communication with the controller, and wherein the controller comprises instructions to actuate the health-monitoring apparatus in response to a signal the controller receives from the one or more auxiliary sensors.

9. The health-monitoring a system of claim 1, wherein the assessment of the user's health status comprises an analysis of at least one of the following list of clinical observations: speed of user movement, user movement patterns, user posture, swelling beneath the user's eyes, swollen lymphatic glands, visual photoplethysmography, heart rate, moles, skin growths, body shape, skin coloration, sclera coloration, degree of transparency of a lens in the user's eye, hair loss, breathing rate, time in front of the at least one camera, and body shape.

10. The health-monitoring system of claim 1, wherein the controller further comprises a graphics processing unit, wherein the graphics processing unit comprises instructions for performing a plurality of nonlinear transformations on the graphic data thereby converting the graphic data to obfuscated graphic data which is unrecognizable by a human viewer and which retains a feature recognizable by the algorithm, and wherein the non-transitory computer-readable media comprises instructions for applying a algorithm to create an analysis of the obfuscated graphic data and an assessment of a user's health status.

11. The health-monitoring system of claim 10, wherein the obfuscated graphic data is created using one or more of the following techniques:
deep convolution;
compressed sensing obfuscation using sparse basis expansion and discarding basis functions; and
block-chain based obfuscation, wherein difference hashing between frames is secured via blockchain.

12. The health-monitoring system of claim 10, further comprising a light source, wherein the light source directs light toward an angle of view of the at least one camera.

13. The health-monitoring system of claim 12, wherein the light source provides light which consists of one or more of the following types of light: visible light, high color temperature light, infrared light, structured light, and modulated light.

14. The health-monitoring system of claim 10, wherein the at least one camera comprises at least one of a 3D time of flight camera, a stereoscopic camera, an infrared thermal imaging camera, a video camera, a structured light 3D scanner, and a still image camera.

15. The health-monitoring system of claim 10, wherein the one or more cameras is disposed within a diagnostic tool, wherein the diagnostic tool comprises of one or more of an otoscope, an ophthalmoscope, an endoscope, a laparoscope, a laryngoscope, a colposcope, a hysteroscope, a bronchoscope, a pharyngoscope, a laparoscope, and a dental tool.

16. The health-monitoring system of claim 10, wherein the at least one camera is disposed within a fixture.

17. The health-monitoring system of claim 16, wherein the fixture comprises a partially silvered mirror, wherein the mirror is disposed in front of a lens of the at least one camera.

18. The health-monitoring system of claim 10, further comprising one or more auxiliary sensors.

19. The health-monitoring system of claim 18, wherein the one or more auxiliary sensor is in electronic communication with the controller, and wherein the controller comprises instructions to actuate the health-monitoring apparatus in response to a signal the controller receives from the one or more auxiliary sensors.

20. The health-monitoring system of claim 10, wherein the assessment of the user's health status comprises an analysis of at least one of the following list of clinical observations: speed of user movement, user movement patterns, user posture, swelling beneath the user's eyes, swollen lymphatic glands, visual photoplethysmography, heart rate, moles, skin growths, body shape, skin coloration, sclera coloration, hair loss, breathing rate, time in front of the camera, and body shape.
